(19) United States
(12) Patent Application Publication
(10) Pub. No.: US 2002/0164390 A1
(43) Pub. Date: Nov. 7, 2002

(54) Compositions containing an active fraction isolated from tricholoma conglobatum and methods of use

(76) Inventor: Kin-Ping Wong, Fresno, CA (US)

Correspondence Address: McCutchen Doyle Brown & Enersen LLP, Suite 1800, Three Embarcadero Center, San Francisco, CA, 94111-4067, US

(21) Appl. No.: 10/021,820

(22) Filed: Dec. 13, 2001

Related U.S. Application Data
(60) Provisional application No. 60/255,563, filed on Dec. 13, 2000.

(51) Int. Cl. A61K 38/16

(52) U.S. Cl. 424/780; 514/2

(57) ABSTRACT

This invention provides processes for isolating pharmaceutically active extracts, fractions and compounds from the mushroom Tricholoma Conglobatum. In one aspect, a homogenate of Tricholoma Conglobatum is extracted from a fraction isolated by column chromatography. It has an optical absorbance between about 210 nm and about 350 nm and is not heat stable. The active compound, having a molecular weight from about 18 to about 20 kD on SDS-PAGE gel, in the extract is further provided by this invention. A second active fraction isolated from the homogenate of Tricholoma Conglobatum can be further processed to separate into two additionally active compounds each of which has characteristics consistent with that of a protein. The fractions, compounds and extracts are useful to inhibit the growth of endothelial cells and to inhibit vascularization in a tissue. Methods of treating various diseases, including cancer, are also provided herein.

### CROSS-REFERENCE TO RELATED APPLICATIONS

This application claims the benefit under 35 U.S.C. §119(e) of U.S. Provisional Application No. 60/255,563, filed Dec. 13, 2000, the content of which is hereby incorporated by reference in its entirety for all purposes.

### FIELD OF THE INVENTION

The present invention is in the field of pharmaceuticals. In particular, it is related to the field of anti-angiogenic pharmaceuticals for the prevention and treatment of disease.

### BACKGROUND

Angiogenesis is the process through which new vascular structures arise by outgrowth from pre-existing capillaries, in this process, endothelial cells become detached from the basement membrane as proteolytic enzymes degrade this support. These cells then migrate out from the parent vessel, divide, and form into a newly differentiated vascular structure (Risau, (1997) Nature 386:671-674; Wilting et al., (1995) Cell. Mol. Biol. Res. 41(4):219-232). A variety of different biological factors have been found to function in controlling blood vessel formation (Bussolino et al., (1997) Trends in Biochem. Sci. 22(7):251-256; Folkman and D'Amore, (1996) Cell 87:1153-1155). These include proteins with diverse functions such as growth factors, cell surface receptors, proteases, protease inhibitors, and extracellular matrix proteins (Achen and Stacker, (1998) Int. J. Exp. Pathol. 79:255-265; Devalaraja and Richmond, (1999) Trends in Pharmacol. Sci. 20(4):151-156; Hanahan, (1997) Science 277:48-50; Maisonpierre et al., (1997) Science 277:55-60; Sun et al., (1996) Cell 87:1171-1180; Sato et al., (1995) Nature 376:70-74; Mignatti and Rifkin, (1996) Enzyme Protein 49:117-137; Pintucci et al., (996) Semin Thromb l-Iemost 22(6)517-524; Vernon and Sage, (1995) Am. J. Pathol. 147(4):873-883; Brooks et al., (1994) Science 264:569-571; Koch et al., (1995) Nature 376:51-519). The complexity of the angiogenic process and the diversity of the factors that control its progression provide a useful array of points for therapeutic intervention to control vascular formation in vivo.

Angiogenesis normally occurs in a carefully controlled manner during embryonic development, during growth, and in special cases such as wound healing and the female reproductive cycle (Wilting and Christ, (1996) Naturwissenschaften 83:153-164; Goodger and Rogers, (1995) Microcirculation 2:329-343; Augustin et al., (1995) Am. J. Pathol. 147(2):339-351). Some of the important steps in the process of angiogenesis are: 1) growth factor (i.e. vascular endothelial growth factor, VEGF) signaling; 2) matrix metalloproteinases (MMP) and VEGF receptor interaction; 3) endothelial cell migration to site of growth factor signaling; 4) endothelial cell tubule formation. Pathological angiogenesis plays a central role in a number of human diseases including tumor growth and metastatic cancer, diabetic retinopathy, rheumatoid arthritis, and other inflammatory diseases such as psoriasis (Folkman, (1995) Nature Med. 1(1):27-31; Polverini, (1995) Rheumatology 38(2):103-112; Healy et al., (1998) Hum. Reprod. Update 4(5):736-396). In these cases, progression of disease is driven by persistent unregulated angiogenesis. For example, in rheumatoid arthritis, new capillary blood vessels invade the joints and destroy the cartilage. In diabetic retinopathy, capillaries in the retina invade the vitreous, bleed and cause blindness. Significantly, tumor growth and metastasis are angiogenesis dependent. Most primary solid tumors go through a prolonged avascular state during which growth is limited to approximately 1-2 mm in diameter. Up to this size, tumor cells can obtain the necessary oxygen and nutrient supply by passive diffusion. These microscopic tumor masses can eventually switch on angiogenesis and recruit surrounding blood vessels to begin sprouting capillaries that vascularize the tumor mass, providing the potential for continuing expansion of the tumor and metastasis of malignant cells to distant location. Although significant progress has been made in understanding the biological events that occur during pathological angiogenesis, there are presently no effective pharmaceutical compounds that are useful for controlling angiogenesis in vivo. Thus, effective therapies capable of controlling angiogenesis have the potential to alleviate a significant number of human diseases.

Traditionally, pharmaceutical compounds have been developed by screening synthetic chemical compounds for desirable pharmaceutical properties and then testing them for toxicity and effectiveness in vivo. Compounds selected this way frequently have toxic side effects in vivo and this approach has not been successful in developing effective angiogenesis inhibitors for disease therapy. More recently, techniques of molecular biology have been applied to develop angiogenesis inhibitors. Protein inhibitors of angiogenesis such as angiostatin (O'Reilly et al., (1994) Cell 79(2):315-328) and endostatin (O'Reilly et al., (1997) Cell 88(2):277-285), that control vascular formation in experimental models have been discovered. Nevertheless, such protein therapeutics are expensive to produce and have been found to be difficult to formulate and deliver in subjects. At present, protein angiogenesis inhibitors have yet to be developed into therapeutic pharmaceuticals for disease patients. Thus, there exists a need for therapeutic compounds that can be safely administered to a patient and be effective at inhibiting the pathological growth of vascular endothelial cells. The present invention provides compositions and methods that are useful for this purpose and provides related advantages as well.

### DISCLOSURE OF THE INVENTION

This invention provides processes for isolating pharmaceutically active extracts, fractions and compounds from the mushroom Tricholoma Conglobatum. In one aspect, the invention provides a process for isolating two extracts, ETCa and ETCb, having pharmaceutical and anti-angiogenic activity. The invention further provides a process for isolating a pharmaceutically active compound designated ATC07α, having a molecular weight from about 18 to about 20 kD on SDS-PAGE gel. In another aspect, the invention provides a process for isolating a fraction, designated ATC07β, by column chromatography. ATC07β has an optical absorbance between about 210 nm to about 350 nm. ATC07β is heat unstable. ATC07β can be further processed to yield ATC07β1 and ATC07β2. ECTa, ECTb, ATC07α, ATC0β, ATC07β1 and ATC07β2 are further provided by this invention. Processes for obtaining the extracts, fractions, and compounds, are further provided herein.

This invention provides methods for inhibiting the growth of endothelial cells by delivering to the cells an effective amount of an extract, fraction or compound of the invention. This invention also provides a method of inhibiting vascularization in a tissue by delivering to the tissue an effective amount an extract, fraction or compound of the invention. Methods of treating various diseases, including cancer, are also provided herein.

### BRIEF DESCRIPTION OF THE FIGURES

FIG. 1 depicts exemplary processes of this invention. In one aspect, the figure depicts procedures for isolating the pharmaceutically active extracts designated ETCa and ETCb, from Tricholoma Conglobatum that possess anti-angiogenic activity. ETCa and ETCb are useful as food and health supplements. ETCa is the pharmaceutically active extract isolated after homogenizing in phosphate buffered saline (“PBS”) (pH 7.0), filtering the homogenate through two layers of Miracloth, and lyophilizing the extract, thereby concentrating the extract. ETCb is obtained by fractionization of the extract that yields ECTa. However, instead of lyophilizing the extract, it is precipitated using ammonium sulfate and 35-70% fraction which is most active is isolated This fraction is dialyzed against 20 mM potassium phosphate (pH 7.5, 3500 kD cutoff) overnight at 4° C. and lyophilized. All extracts, fractions, pellets, and supernatants are assayed for anti-angiogenic activity using the CPAE assay after each step to ensure that no anti-angiogenic activity is lost or misplaced.

FIG. 1 further depicts a procedure for isolation of ATC07α. It is isolated after chromatographic separation using a Sephacryl S-200 (“S-200”) column. ACT70β is eluted using the same Sephacryl S-200 column along with a sodium chloride (NaCl) gradient.

FIG. 1 further provides procedure for isolating ATC07β1 and ATC07β2. These are recovered following further chromatographic separation of ACT70β in a FPLC analysis on a Hydroxyapatite column using either a salt gradient, phosphate gradient, and/or a pH gradient. See FIG. 9.

The extractions can also be performed using water or Tris buffer as well as phosphate buffered solution (PBS), all at concentrations ranging from 5 mM to 1M and pH 6.8 to 8.8.

FIG. 2 is a graph showing activity of the fractions as they are isolated from crude extract off the MONO-Q FPLC. Column conditions are: buffer (20 mM potassium phosphate, pH=7.5); flow rate (2 mL/minute); detection (280 nm OD, sensitivity=0.5); temperature 25° C. and gradient elution at 0 to 1 M sodium chloride. Activity was carried out on Calf Pulmonary Arterial Endothelial (“CPAE”) cell culture assays. Two active fractions, α and β were obtained.

FIG. 3 is a graph showing activity after purification with S-200 FPLC (fast protein liquid chromatography). Bio-Rad Protein Assay and ECC (endothelial cell culture) was carried out on each fraction. Fraction #36 has anti-angiogenesis activity and had pure protein component. This fraction was designated ATC07α.

FIG. 4 is a graph showing concentration dependence of inhibition activity of ATC07α. Angiogenesis inhibition activity was achieved and determined by endothelial cell culture (ECC) assay.

FIG. 5 shows the results from the Bio-Rad protein and carbohydrate assays, together with inhibition assay of ACT70α.

FIG. 6 is the spectrophotometer (SHIMADZU UV-1601) scan for ATC07α. There is a distinct peak around 280 nm. The absorbance ratio for 280 nm/260 nm is 1.8, which indicates that the sample is a protein.

FIG. 7 is the Circular Dichroism (CD) spectrum of ATC07α. There is a near ultraviolet CD region (350-300 nm characteristic of tertiary structure of proteins, and a far ultraviolet CD region (260-200 nm) characteristic of a predominantly, structured protein.

FIG. 8 is the FPLC MONO Q separation of DEAE Sephadex peak β (0 to 350 mM NaCI gradient). DEAE Sephadex fractions were pooled, dialyzed against water and lyophilized. 35 mg dry residue was dissolved in 4 ml 20 mM Tris HCl, pH 7.4 and applied to a 10/10 MONO Q column. Elution was performed using a 0-350 mM NaCl gradient. All fractions were assayed using the CPAE assay. Active fractions, Beta, were pooled, dialyzed against water and lyophilized.

FIG. 9 is the Hydroxyapatite Chromatography run of MONO Q Beta. Lyophilized MONO Q Beta, 35 mg, was dissolved in 4 ml 10 mM potassium phosphate pH 7.2 and applied to a Hydroxyapatite column (20 ml). Elution was performed using a 0-500 mM NaCl gradient. Two active regions were found: Beta 1 (“β1”) and Beta 2 (“β2”). Fractions for each region were pooled, dialyzed against water and lyophilized.

FIG. 10 is a Hydroxyapatite Chromatography rerun of Beta 2 (“β2”). 2.7 mg of this fraction was dissolved in 2 ml 1 mM potassium phosphate pH 6.8 and applied to a Hydroxyapatite column (5 ml). Elution was performed using a 1-300 mM phosphate gradient.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The practice of the present invention employs, unless otherwise indicated, conventional techniques of molecular biology (including recombinant techniques), microbiology, organic chemistry, cell biology, biochemistry and immunology, which are within the skill of the art. Such techniques are explained fully in the literature.

Definitions

As used herein, certain terms may have the following defined meanings.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are well known in the art.

The term “isolated” means separated from constituents, cellular and otherwise, in which the compound is normally associated with in nature.

A “subject” or “host” is a vertebrate, preferably an animal or mammal, more preferably a human patient. Mammals include, but are not limited to, murines, simians, human patients, farm animals, sport animals, and pets.

The terms “cancer,” “neoplasm,” and “tumor,” used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

As used herein, “inhibit” means to stop, delay or slow the growth, proliferation or cell division of endothelial cells or the formation of blood vessels in tissue. Methods to monitor inhibition include, but are not limited to endothelial cell proliferation assays, measurement of the volume of a vascular bed by determination of blood content and quantitative determination of the density of vascular structures. When the culture is a mixture of cells, neovascularization is monitored by quantitative measurement of cells expressing endothelial cell specific markers such as angiogenic factors, proteolytic enzymes and endothelial cell specific cell adhesion molecules.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results. This amount may be the same or different from a prophylactically effective amount, which is an amount necessary to prevent onset of disease or disease symptoms. An effective amount can be administered in one or more administrations, applications or dosages.

Applicant has identified processes for isolating pharmaceutically active extracts, fractions and compounds, designated herein as ETCa, ETCb, ATC07α, ATC07β, ATC07β1 and ATC07β2, from the mushroom Tricholoma Conglobatum. ETCa is the active extract isolated after homogenizing and filtration of a hot “tea” containing an effective amount of Tricholoma Conglobatum. The filtrate can be concentrated by methods well known in the art, e.g., lyophilization. Extract ETCb is obtained by filtration of ETCa and precipitation using methods well known in the art, e.g., using ammonium sulfate. The 35-70% fraction appears to be most active and contains the extract ETCb. The extract can be dialyzed against 20 mM potassium phosphate (pH 7.5, 3500 kD cutoff) overnight at 4° C. and lyophilized.

In one aspect, an effective amount of Tricholoma Conglobatum is homogenized in an effective amount of phosphate buffered saline (PBS) and filtered to collect the supernatant, designated ETCa herein. This is concentrated by precipitation and dialysis to obtain ETCb which is further processed to obtain a fraction, ATC07α, having an optical absorbance at about 210 nm to about 350 nm.

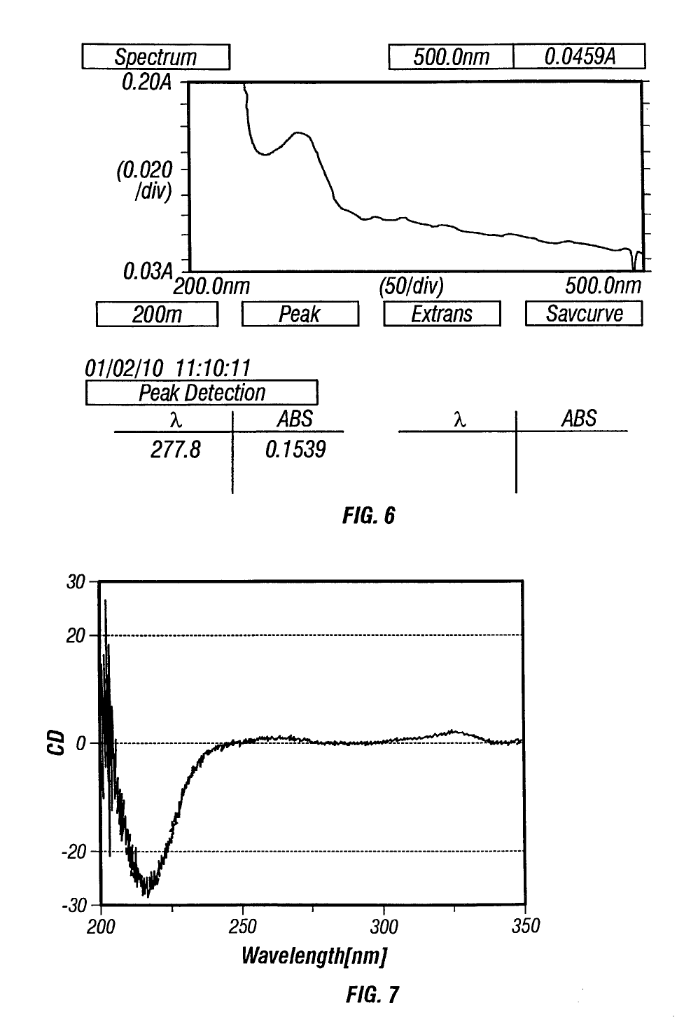

In an alternative aspect, an effective amount of Tricholoma Conglobatum is homogenized in an effective amount of PBS and filtered and the supernatant is collected. The supernatant is precipitated with an effective amount of ammonium sulfate and the 35% to 70% cut is collected. This is concentrated by dialysis against an effective amount of potassium phosphate and the active fractions are purified by column chromatography. In a separate aspect, a fraction designated ATC07α is isolated. ATC07α has an optical absorbance between about 210 nm and about 350 nm and is isolated from a homogenate of Tricholoma Conglobatum. In a further aspect, the active fraction ATC07α has an optical absorbance of about 270 nm to about 290 nm. In a yet further aspect, the pharmaceutically active fraction has an optical absorbance after chromatography of about 280nm. FIGS. 5 through 7 depict further characterization of ATC07α. ATC07α is a protein that is not heat stable but shows almost 100% inhibition of endothelial cell proliferation. Anti-angiogenic activity is concentration dependent. Acid and neutral carbohydrates are also present, but in much smaller concentrations. ATC07Q has a molecular weight of about 18 to about 20 kD as determined on SDS-PAGE.

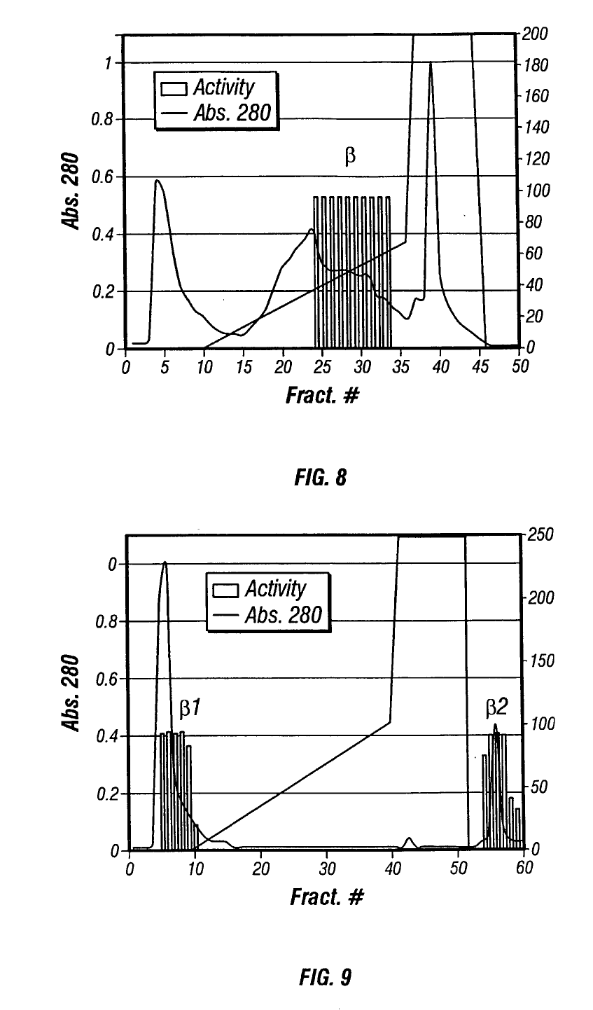

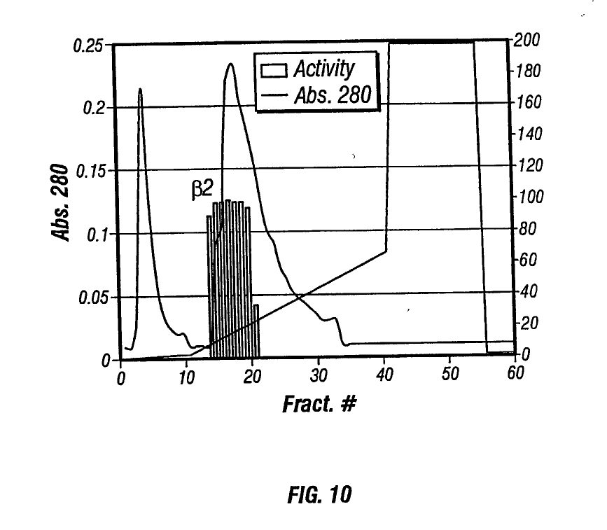

The second active fraction, ATC07β, is purified by elution off the Mono-Q ion exchange column with 0.3M NaCl. The eluate is further purified by running it through a Sephacryl S-200 column or its equivalent at a rate of 2 mL/min. Further chromatographic separation in a FPLC analysis on a Hydroxyapatite column using either a NaCl gradient, phosphate gradient, and/or a pH gradient yields the presence of ATC07β1 and ATC07β2. The active fractions elute at about 6 to 11 (ATC07β1) and about 5 to 60 (ATC07β2), see FIG. 8.

The inventor has also discovered that the extracts, fractions and compounds inhibit endothelial cell growth and possess anti-angiogenic properties. In accordance with these findings, this invention provides methods for inhibiting the growth of endothelial cells by delivering to the cells a growth inhibitory amount of an extract, a fraction, a compound or composition comprising one or more of the same (also collectively referred to herein as “drug”). This invention also provides methods of inhibiting vascularization in a tissue by delivering to the tissue an anti-vascularization amount of an extract, a fraction, a compound or composition comprising one or more of the same.

This method can be practiced in vitro or in vivo. When practiced in vitro, endothelial cells or vascularized tissue are cultured under conditions well known to those skilled in the art, e.g., as exemplified below. The cells and/or tissue can be from an established cell line or cultured from a biopsy sample obtained from a subject. The extract, fraction or compound is then directly added to the culture medium or delivered as a component of a pharmaceutical composition.

Not every therapy is effective for each individual and therefore, an in vitro assay to gauge efficacy for each patient would be advantageous. The present method provides these means to determine whether compositions or therapies will treat individual's specific disease related to pathological proliferation of endothelial cells. For example, a tissue biopsy is isolated from the patient and contacted with an effective amount of a pharmaceutically active extract, fraction or compound or therapy as defined herein and under conditions effective for growth and proliferation of the cells. Inhibition of growth of the pathological cells as determined by conventional procedures, e.g., the CPAE assay described herein, indicates that the inventive extracts, fractions, or compounds and/or therapies may effectively treat the patient.

Angiogenesis or the formation of new vasculature is a fundamental process by which new blood vessels are formed. It participates in essential physiological events, such as reproduction development and wound healing. Under normal conditions, angiogenesis is highly regulated. However, many diseases are driven by persistent unregulated angiogenesis. In rheumatoid arthritis, new capillary blood vessels invade the joints and destroy the cartilage. In diabetic retinopathy, new capillaries in the retina invade the vitreous, bleed, and cause blindness. Tumor growth and metastasis are angiogenesis-dependent. Most primary solid tumors go through a prolonged state of avascular, and apparently dormant, growth in which the maximum size attainable is ˜1-2 mm in diameter. Up to this size, tumor cells can obtain the necessary oxygen and nutrient by simple passive diffusion. These microscopic tumor masses can eventually switch on angiogenesis by recruiting surrounding mature host blood vessels to begin sprouting new blood vessel capillaries which grow toward, and eventually infiltrate the tumor mass, thus setting in motion the potential for relentless expansion of tumor mass and hematogenous metastatic spread as well. The angiogenic switch was initially hypothesized to be triggered by the ectopic production and elaboration by tumor cells of a growth factor called “tumor angiogenesis factor” (TAF).

This invention also provides a method of treating a disorder associated with pathological neovascularization or endothelial cell growth in a subject by administering to the subject a therapeutically effective amount or a growth inhibitory amount of an extract, fraction or compound of this invention, i.e., ETCa, ETCb, ATC07α, ATC07β, ATC07β1and/or ATC07β2-2 or a pharmaceutically acceptable derivative, salt or prodrug thereof. As used in this context, to “treat” means to alleviate the symptoms associated with pathological neovascularization and/or endothelial cell growth as well as the reduction of neovascularization or endothelial cell growth. Such conditions include, but are not limited to arthritic conditions, neovascular-based dermatological conditions, diabetic retinopathy, Karposi's Sarcoma, age-related macular degeneration, restenosis, telangectasia, glaucoma, keloids, corneal graft rejection, wound granularization, angiofibroma, Osler-Webber Syndrome, myocardial angiogenesis, and scleroderma. Exemplary arthritic conditions are selected from the group consisting of rheumatoid arthritis, psoriatic arthritis and osteoarthritis. For the treatment of cancers and solid tumors, to “treat” includes inhibition of the growth of blood vessels resulting in a lack of nutrients for the tumors and/or cancer cells needed by the tumor for its growth. Tumors and growths will decrease in size and possibly disappear. Administration for the treatment of arthritic conditions will result in decreased blood vessel formation in cartilage, specifically joints, resulting in increased mobility and flexibility in these regions. For the treatment of psoriasis, administration will reduce dermatological symptoms such as scabbing, flaking and visible blood vessels under the surface of the skin. In diabetic retinopathy, administration of ETCa, ETCb, ATC0α, ATC07β, ATC07β1 and/or ATC07β2 will reduce the formation of extraneous blood vessels in the retina, resulting in unobstructed vision. In the treatment of Karposi's Sarcoma, administration of ETCa, ETCb, ATC0α, ATC07β, ATC07β1 and/or ATC07β2 inhibit the growth and/or further formation of blood vessels, thereby inhibiting the formation of lesions and/or tumors that arise.

When an extract, a fraction, a compound or composition comprising one or more of the same is administered to a subject such as a mouse, a rat or a human patient, it can be added to a pharmaceutically acceptable carrier and systemically, orally, transdermally or topically administered to the subject. Therapeutic amounts can be empirically determined and will vary with the pathology being treated, the subject being treated and the toxicity of the form of the active fraction used in the therapeutic method. Active extract, fractions, compounds or compositions comprising one or more of the same in various forms can be delivered orally, intravenously, intraperitoneally, or transdermally. When delivered to an animal, the method is useful to further confirm efficacy of the extract, fraction, compound or composition comprising one or more of the same, for the disease sought to be treated.

As an example of an animal model, groups of nude mice (Balb/c NCR nu/nu female, Simonsen, Gilroy, Calif.) are each subcutaneously inoculated with about 10⁵ to about 10⁹ pathological cells as defined herein. When the graft is established, the extract, fraction, compound or composition comprising one or more of the same, is administered, for example, by subcutaneous injection around the graft. Measurements to determine reduction of graft size are made in two dimensions using venier calipers twice a week.

The MRL/lpr mice (MRL/MpJ-Fas^{1pr}) from Jackson Labs (Maine) are useful to test or monitor efficacy in arthritic conditions. A positive therapeutic benefit includes reduced swelling of the joints and hindlegs of animals and reduced cartilage degradation which can be monitored by X-ray.

Administration in vivo can be effected in one dose, multiple doses, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The extracts, fractions, compounds, compositions and pharmaceutical formulations of the present invention can be used in the manufacture of medicaments, food and health supplements, and for the treatment of humans and other animals by administration in accordance with conventional procedures, such as an active ingredient in pharmaceutical compositions.

The extract, fraction, compound or composition comprising one or more of the same, compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to a drug of the present invention, the pharmaceutical compositions can also contain other pharmaceutically active compounds or a plurality of compounds of the invention.

Active extracts, fractions, compounds or compositions comprising one or more of the same, are administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

It will be appreciated that appropriate dosages of the extract, fraction, compound or composition comprising one or more of the same, of the invention may depend on the type and severity and stage of the disease and can vary from patient to patient. Determining the optimal dosage will generally involve the balancing of the level of therapeutic benefit against any risk or deleterious side effects of the treatments of the present invention.

Ideally, an active extract, fraction, compound or composition comprising one or more of the same, should be administered to achieve peak concentrations of the active compound at sites of disease. This may be achieved, for example, by the intravenous injection of the fraction or composition, optionally in saline, or orally administered, for example, as a tablet, capsule or syrup containing the active ingredient. Desirable blood levels of the drug may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

While it is possible for the drug ingredient to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

The extract, fraction, compound or composition comprising one or more of the same, can also be used in combination with other compounds or compositions with known or suspected anti-angiogenic agents such as shark cartilage, tyrosphingosine, sphingosine, and other anti-angiogenic agents. They also can be combined with immune enhancing agents, e.g., interferons such as interferon-α and traditional cancer therapies, e.g., radiation and the like.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, paste, gel, spray, aerosol or oil. Alternatively, a formulation may comprise a patch or a dressing such as a bandage or adhesive plaster impregnated with active ingredients and optionally one or more excipients or diluents.

For diseases of the eye or other external tissues, e.g., mouth and skin, the formulations are preferably applied as a topical ointment or cream containing the active ingredient. When formulated in an ointment, the drug may be employed with either a paraffinic or a water-miscible ointment base. Alternatively, the drug ingredients may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound that enhances absorption or penetration of the drug ingredient through the skin or other affected areas. Examples of such dermal penetration enhancers include dimethylsulfoxide and related analogues.

The oily phase of the emulsions of this invention may be constituted from known ingredients in any known manner. While this phase may comprise merely an emulsifier (otherwise known as an emulgent) it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient.

Formulations suitable for parenteral administration include aqueous and nonaqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other micro particulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above recited, or an appropriate fraction thereof, of a drug ingredient.

It should be understood that in addition to the ingredients particularly mentioned above, the formulations of this invention may include other agents conventional in the art having regard to the type of formulation in question, for example, those suitable of oral administration may include such further agents as sweeteners, thickeners and flavoring agents.

The extract, fraction, compound or composition comprising one or more of the same, (“referred to herein as “drug”) may also be presented for the use in the form of veterinary formulations, which may be prepared, for example, by methods that are conventional in the art.

This invention further provides a method for screening for a therapeutic agent for inhibiting neovascularization or endothelial cell growth. The screen requires:

(a) contacting the agent with a suitable cell or tissue sample;

(b) contacting a separate sample of the suitable cell or tissue with a therapeutically effective amount of a drug this invention or a pharmaceutically acceptable composition containing the same; and

(c) comparing the growth of the sample of step (a) with the growth of the sample of step (b), and wherein any agent of step (a) that inhibits the growth to the same or similar extent as the sample of step (b) is a therapeutic agent for inhibiting neovascularization or the growth of endothelial cells. Optionally, the samples may contain one or more agents selected from the group consisting of anti-angiogenic, anti-tumor and immune enhancing. Further, they sample may be exposed to other traditional therapies, e.g., radiation and the like.

As used herein, a suitable sample intends any sample that contains endothelial cells or vascularized tissue. The method can be practiced in vitro or in vivo as described herein.

A kit for treating a disorder associated with pathological neovascularization or endothelial cell growth in a subject, also is provided by this invention. The kit includes a therapeutically effective amount of an extract, a fraction, a compound or composition comprising one or more of the same, and instructions for use. The kit is useful to treat disorders selected from the group consisting of arthritic conditions, neovascular-based dermatological conditions, diabetic retinopathy, Karposi's Sarcoma, age-related macular degeneration, restenosis, telangectasia, glaucoma, keloids, corneal graft rejection, wound granularization, angiofibroma, Osler-Webber Syndrome, myocardial angiogenesis, scleroderma, rheumatoid arthritis, psoriatic arthritis and osteoarthritis.

The following examples are intended to illustrate, but not limit the invention.

### EXAMPLES

Materials

The following materials were used in the methods described below.

Tricholoma Conglobatum was obtained from Q. G. Mushroom Inc. in Fresno, California. The fresh Tricholoma fruiting body was homogenized and extracted. The whole extraction process was carried out at 4° C., and the extraction was kept at −20° C.

The following reagent chemicals used were in the examples described herein. However, it is to be understood, although not always explicitly stated that the reagents described herein are merely exemplary and that equivalents of such are well known in the art. The following are examples and equivalents thereof are within the scope of this invention: Heparin (Sigma); Glucose (Sigma); Potassium Phosphate, Monobasic, (Sigma); Sodium Chloride (Sigma); Sodium Acetate (Sigma); Ammonium Sulfate (Sigma); Hydrochloric Acid (VWR Scientific); Phosphatase Substrate (Sigma); Sephacryl S-200 HR (Sigma); Mono-Q Ion Exchange Column (Sigma); and TritonX-100 (Sigma). All the chemicals used were Analytical Chemical Grade.

### Example 1

Isolation and Purification

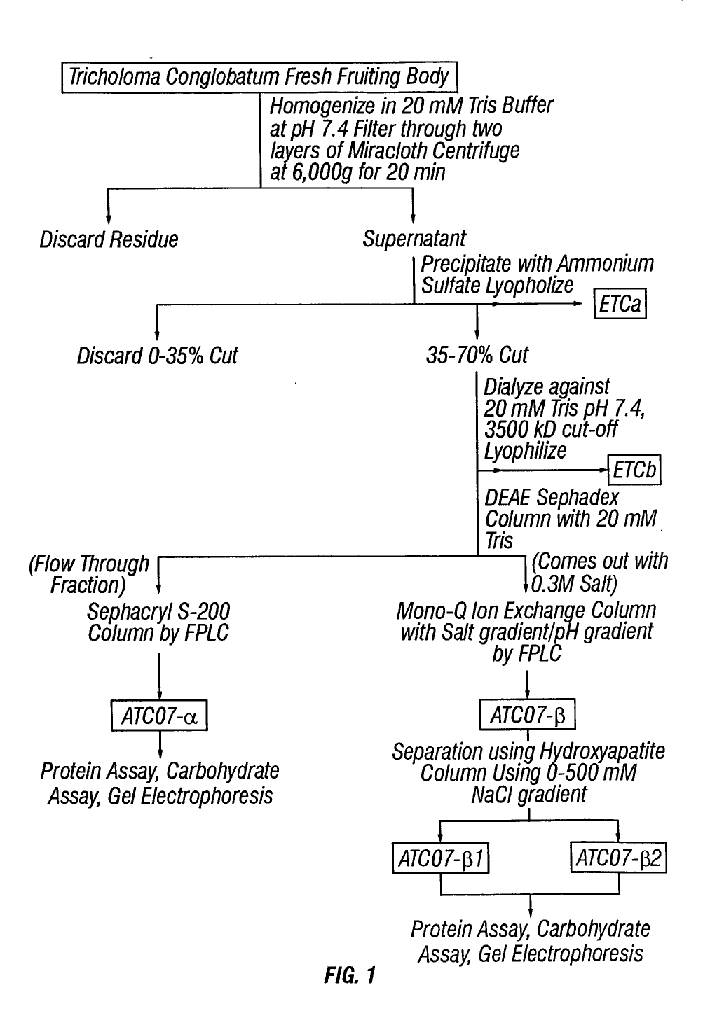

This invention provides several embodiments of the process for preparing a biologically active fraction from a composition containing the mushroom of Tricholoma Conglobatum. See FIG. 1.

In one aspect, an effective amount of Tricholoma Conglobatum is homogenized in an effective amount of phosphate buffered saline (PBS) and filtered to collect the supernatant, designated ETCa herein. This is concentrated by precipitation and dialysis to obtain ETCb.

In an alternative embodiment, an fraction having an optical absorbance of between about 210 nm and 350 nm is isolated. In a further aspect, the fraction has an absorbance from about 270 nm to about 290 nm. In a yet further aspect, fraction's absorbance is around 280 nm.

In a further aspect, Tricholoma fruiting body was extracted with doubly distilled (dd) water and phosphate buffered saline (PBS, pH=7.0). The supernatant extract was precipitated at different saturation degrees of ammonium sulfate. See FIG. 1 and Tables 1 and 2, below.

TABLE 1
Extraction of Tricholoma Using Different Solutions

| | Inhibition of Crude Extract | Amount of |
|---|---|---|
| Extraction Solution | [% inhibition/mg/mL sample] | Crude Extraction* |
| Double distilled water | 75 | 4.22 g |
| PBS (pH = 7.0) | 98 | 4.38 g |

*Extracted from 200 g fresh fruiting body

TABLE 2
Ammonium Sulfate Fractionation

| Saturation Degree of Ammonium Sulfate | % Inhibition (mg/mL sample) |
|---|---|
| 0-35% cut | 0 |
| 35-70% cut | 98 |

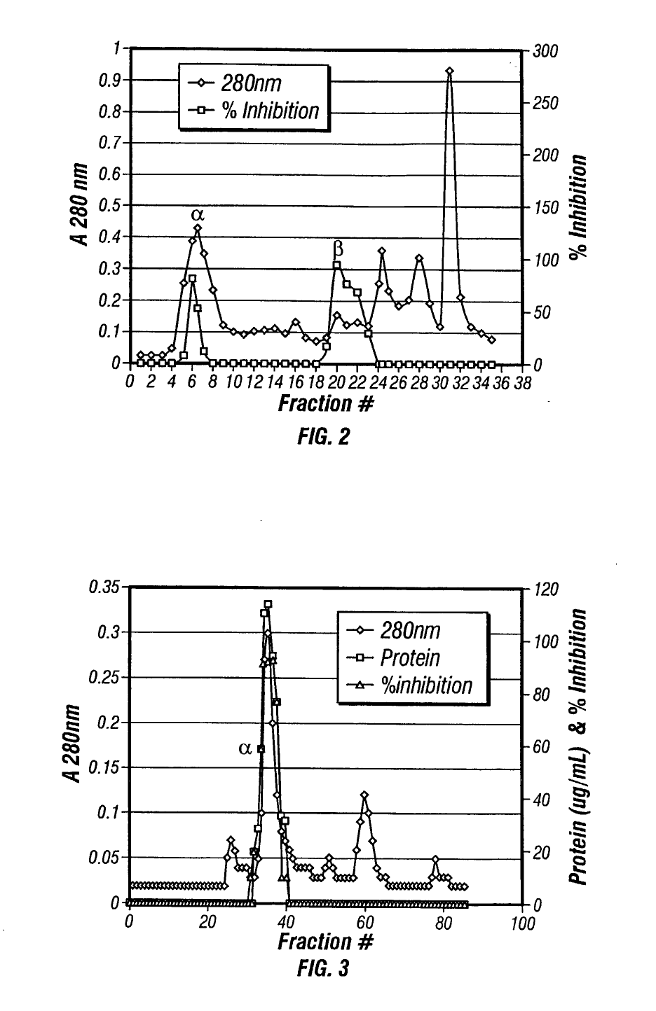

Crude extractions from Tricholoma fruiting body were purified by ion-exchange chromatography and gel filtration chromatography, respectively. See FIG. 1. ECC assay was carried out and the results are shown in FIG. 2.

The active fraction from Mono-Q ion exchange column (Sigma) was put on Sephacryl-200 HR (Sigma). Each fraction (FIG. 1, α and β) from the S-200 column was analyzed by ECC assay. Results are shown in FIG. 3. The S-200 column was run using gel filtration molec weight markers (Sigma). The active fraction ATC07α was taken the flow through of a S-200 column was analyzed by SDS-PAGE gel electrophoresis for molecular weight. Three bands were achieved. All components were in the 18-20 kD range, which was correspondent to the result from S-200 column.

Further chromatographic separation of the β fraction in a FPLC analysis on a Hydroxyapatite column using either a salt gradient, phosphate gradient, and/or a pH gradient yields the presence of ACTO7β1 and ACT07β2. See FIG. 9.

The procedures described herein can also be performed using water or Tris buffer as well as phospate buffered saline (PBS), all at concentrations ranging from 5 mM to 1M and pH 6.8 to 8.8.

### Example 2

Determination of Angiogenesis-Inhibition by Endothelial Cell Culture (ECC) Assay

The assays were carried out according to the procedures of Connally, et al. (1986) Anal. Biochem. 152:136-4 with modifications (Liang and Wong (1999) ANGIOGENESIS: FROM THE MOLECULAR TO INTEGRATIVE PHARMACOLOGY edited by Maradoudakis, Kluwer Academic/Plenum. Publishers, New York). D.T. Connolly et al. (1986) Anal. Biochem. 152:136-140. CPAE (Cardiopulmonary Artery Endothelial Cells, bovine) acquired from American Type Tissue Culture (ATTC) were grown to nearly 95% confluence in MEM-10E. The cells were released from the tissue culture flask with a 0.25% trypsin solution and plated in 24 well tissue culture plates in the same culture medium at a density of 10,000 cell/well. After the plates were cultivated for 8 hours at 37° C. in a 5.0% CO₂ incubator. Assay samples and controls were added. Each sample was loaded in two different wells at 100 μL/well to insure reproducibility. After incubation with the sample for 60 hours, the medium was aspirated, and the number of cells was measured on the basis of the colorimetric measurement of cellular acid phosphatase.

### Example 3

Sample Titration Assay

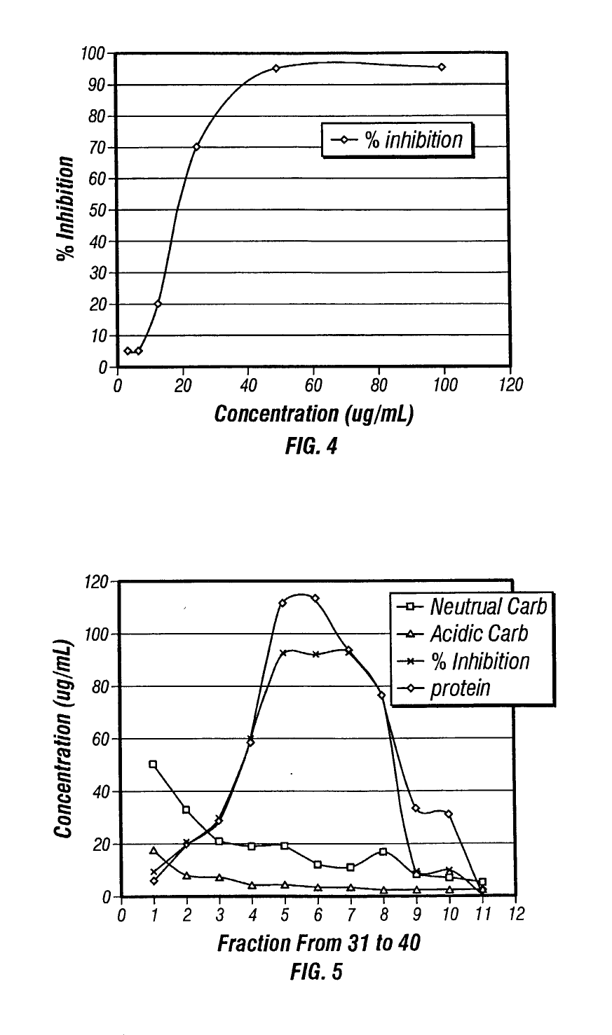

The titration assay was carried out on endothelial cells to confirm that anti-angiogenesis activity from the fraction is dosage related. Samples were made in concentration titrations from 1.0 mg/mL to 0.00625 mg/mL. Different samples were loaded on cells, while a blank was the control. Results are shown in FIG. 4.

### Example 4

Analysis of ATC07α

The protein concentration of ATC07a was determined with the Bio-Rad Bradford protein assay (Bradford, 1976) Anal. Biochem. 72:248-254. An 800 μL sample was mixed with 200 μL of the Bio-Rad (1 to 5 dilution) and then analyzed for absorbency at 595 nm using Shimadzu Spectrophotometer Model UV-1 601. See FIG. 5 for results.

### Example 5

Carbohydrate Assays

Phenol-sulfuric acid reaction was used to determine the presence of neutral carbohydrates (Dubois et al., (1956) Analyt. Chem. 28:350-356). A 200 μL sample was mixed with 1 mL of concentrated sulfuric acid, and heated for 5 min in a boiling water bath, and allowed to cool down to room temperature rapidly. 10 μL of 80% phenol˜water was then added. The mixture was then heated at 100 ° C. for 10 minutes. The absorbency was read at 490 nm. Glucose was used as standard.

A modified carbazol assay was used to test the presence of acidic carbohydrate (Bitter and Muir (1962) Anal. Chem. 4:330-334). A 200 μL of sample was mixed with 1 mL of 0.025 M sodium tetraborate in concentrated sulfuric acid. After thorough mixing, the solution was heated in a boiling water bath for 10 minutes, then cooled down to room temperature rapidly. Then 40 μL of 0.125θ% carbazol in ethanol was added. The solution was heated again at 100° C. for 15 minutes. After cooling down, the absorbency was read at 530 nm. Heparin was used as a standard. See FIG. 5.

### Example 6

Heat Treatment

Samples of ATC07α were prepared with a final concentration of 1.0 mg/mL. The samples were heated at 60° C. and 100 ° C., separately for 5 minutes, 10 minutes and 20 minutes. The control was a sample of ATC07α without any heat treatment. Samples and controls were tested by the ECC assay, described above. The results are shown in Table 3. Heat treatment does affect the inhibition activity, providing additional indication that ATC07α is a protein.

TABLE 3
Angiogenesis Inhibition Activities of ATC07 α Upon Heat Treatment

| Temperature | 5 minutes | 10 minutes | 20 minutes |
|---|---|---|---|
| 60° C. | About 10% | 0% | 0% |
| 100° C. | 0% | 0% | 0% |

### Example 7

Cytolytic/Cytotoxic Assay Calf Pulmonary Arterial Endothelial (CPAE) cells are plated at 10,000 cells per well in 24 well culture plates. After growth incubation at 37° C., 5% CO₂ for about 60 hours, a dosage of the sample is added (about 50 μl to about 100 μl) to each sample well and re-incubated for 30 minutes. After incubation, cells are assayed visually under an inverted microscope to detect the presence of cells and through the use of the ECC assay. Both methods are used to detect the presence or absence of endothelial cells in each well. Control cells containing no sample were used and grew normally.

### Example 8

CAM Assay

The chorioallantoic membrane (CAM) assay (Nguyen, M., et al. (1994) Microvas. Res. 47:31-40) is used to determine the efficacy of an active fraction, extract or compound in an in vivo model. Fertilized chicken eggs are obtained from Kings Valley Farms (Kingsburg, Calif.) and incubated at 37° C. in a humidified chamber. Eggs are rotated 180 once daily for four days at which time windows are cut in the shells as follows: First, eggs are swabbed with 95% ethanol and a small hole placed in the blunt end of the egg which will collapse the air sack present there. The membrane falls away from the shell when a 1 cm² “window” is removed from the shell using a hacksaw (25 teeth/in). Sterilized Howard's Ringer solution is used to wash away any excess shell fragments. The eggs are then sealed with cellophane tape and returned to the incubator. After 4-7 days, the window is reopened and a sterile cover slip is placed to serve as the reference point. For testing, a hole is created with a sterile needle between blood vessels in the CAM and a small amount of sample was applied to the hole, which is then resealed. Lactose is used as the control. Observations are made daily for 4 days for any inhibition of blood vessel development on the CAM.

### Example 9

MMP Assay

P.C. Brooks, et. al. (1996) in “Localization of matrix metalloproteinase MMP-2 to the surface of invasive cells by interaction with integrin αvβ3,” Cell 85:683-93 describes an in vitro assay on matrix metalloproteinase and αv/β3 integrin interaction. The effects of the experimental sample on the MMP-2/αvβ3 integrin complex determines if the sample's mechanism of action involves any disruption of this segment of the angiogenic pathway. This involves testing if the experimental sample can inhibit the interaction of MMP-2 with the αvβ3 integrin. Initially, this is done via an ELISA using antibodies for MMP-2 and testing the binding of these antibodies to the sample. Further studies are pursued if a positive result occurs. TIMP-2 (Tissue Inhibitor of Matrix Metalloprotease-2), a known natural inhibitor of MMP-2, is used as the control.

### Example 10

Endothelial Cell Tubule/Cord Formation Assay Matrigel (60 μl of 10 mg/ml; Collaborative Lab #35423) is placed in each well of an ice-cold 96-well plate. The plate is allowed to sit at room temperature for 15 minutes then incubated at 37° C. for 30 minutes to permit the matrigel to polymerize. In the mean time, HUVEC are prepared in EGM-2 (Clonetic #CC3162) at a concentration of 2×10⁵ cells/ml. The test compound is prepared at 2× the desired concentration (5 concentration levels) in the same medium. Cells (500 μl) and 2× fraction or compound (500 μl) is mixed and 200 μl of this suspension are placed in duplicate on the polymerized matrigel. After a 24 hour incubation, triplicate pictures are taken for each concentration using a Bioquant Image Analysis system. Drug effect (IC₅₀) is assessed compared to untreated controls by measuring the length of cords/tubules formed and number of junctions. TNP-470 (NSC 642492) and paclitaxel (NSC 125973) are used as reference compounds.

### Example 11

Endothelial Cell Migration Assay

Migration is assessed using the 48-well Boyden chamber and 8 μm pore size collagen-coated (10 μg/ml rat tail collagen; Collaborative Laboratories) polycarbonate filters (Osmonics, Inc.). The bottom chamber wells receive 27-29 μl of DMEM medium alone (baseline) or medium containing chemo-attractant (bFGF, VEGF or Swiss 3T3 cell conditioned medium). The top chambers receive 45 μl of HUVEC cell suspension (1×10⁶ cells/ml) prepared in DMEM+1% BSA with or without the fraction or compound. After a 5 hour incubation at 37° C., the membrane is rinsed in PBS, fixed and stained in Diff-Quick solutions. The filter is placed on a glass slide with the migrated cells facing down and cells on top are removed using a Kimwipe. The testing is performed in 4-6 replicates and five fields are counted from each well. Negative unstimulated control values are subtracted from stimulated control and fraction or compound treated values and data is plotted as mean migrated cell ±S.D. IC₅₀ is calculated from the plotted data. TNP-470 (NSC 642492) and paclitaxel (NSC 125973) are used as reference compounds.

Although the foregoing invention has been described in some detail by way of illustration and example for purposes of clarity of understanding, it will be apparent to those skilled in the art that certain changes and modifications will be practiced. For example, as is apparent to those of skill in the art, the invention method can be combined with one or more known anti-tumor, anti-angiogenic or immune enhancing therapies and/or compositions, e.g., shark cartilage, tyrosphingosine or sphingosine. Therefore, the description and examples should not be construed as limiting the scope of the invention, which is delineated by the appended claims.

What is claimed is:

1. A heat labile compound having characteristics consistent with that of a protein that inhibits endothelial cell growth and wherein the molecular weight of the compound is from about 18 kD to about 20 kD.

2. A composition comprising the compound of claim 1 and a pharmaceutically acceptable carrier.

3. The composition of claim 2, further comprising an agent selected from the group consisting of anti-angiogenic, anti-tumor or immune enhancing.

4. A process for obtaining an active extract designated ETCa having characteristics consistent with that of a protein that inhibits endothelial cell growth from a composition of Tricholoma Conglobatum, comprising homogenizing an effective amount of Tricholoma Conglobatum in an effective amount of buffer solution and filtering to collect the active extract designated ETCa.

5. A pharmaceutically active extract obtainable from the process of claim 4.

6. A composition comprising the extract of claim 5 and a pharmaceutically acceptable carrier.

7. The composition of claim 6, further comprising an agent selected from the group consisting of anti-angiogenic, anti-tumor or immune enhancing.

8. A process for obtaining an active fraction ETCb from a composition of Tricholoma Conglobatum, comprising the steps of:
a) homogenizing an effective amount of Tricholoma Conglobatum in an effective amount of buffer solution and filtering; and
b) purify by ammonium sulfate fractionization and dialysis to obtain ETCb.

9. A pharmaceutically active extract having characteristics consistent with that of a protein that inhibits endothelial cell growth obtainable from the process of claim 8.

10. A composition comprising the extract of claim 9 and a pharmaceutically acceptable carrier.

11. The extract of claim 10, further comprising an agent selected from the group consisting of anti-angiogenic, anti-tumor or immune enhancing.

12. A process for preparing a biologically active fraction ATC07α from a composition of Tricholoma Conglobatum comprising the steps of:
a) homogenizing an effective amount of Tricholoma Conglobatum in an effective amount of buffer solution and filtering to collect the supernatant;
b) concentrating the supernatant; and
c) isolating a fraction having an optical absorbance at about 210 nm to about 350 nm.

13. A process for preparing a biologically active compound ATC07α from a composition of Tricholoma Conglobatum comprising the steps of:
a) homogenizing an effective amount of Tricholoma Conglobatum in an effective amount of buffer solution and filtering to collect the supernatant;
b) precipitating the supernatant with an effective amount of ammonium sulfate;
c) collecting the 35% to 70% ammonium sulfate fraction;
d) dialyzing against an effective amount of potassium phosphate;
e) purifying the active fractions by ion exchange column chromatography; and
f) obtaining the active compound by chromatography to obtain a biologically active extract having an optical absorbance from about 210 nm to about 350 mn.

14. A compound obtainable by the process of claim 12 or 13.

15. The compound of claim 14, further comprising an effective amount of an agent selected from the group consisting of anti-angiogenic, anti-tumor and immune enhancing.

16. A process for preparing a biologically active compound ATC07β from a composition of Tricholoma Conglobatum comprising the steps of:
a) homogenizing an effective amount of Tricholoma Conglobatum in an effective amount of buffer solution and filtering to collect the supernatant;
b) precipitating the supernatant with an effective amount of ammonium sulfate;
c) collecting the 35% to 70% fraction;
d) dialyzing against an effective amount of potassium phosphate;
e) purifying the active fractions by ion exchange column chromatography to obtain the active fractions ACT70β.

17. A pharmaceutically active fraction obtainable from the process of 16.

18. The fraction of claim 17, further comprising an agent selected from the group consisting of anti-angiogenic, anti-tumor or immune enhancing.

19. The process of claim 16, further comprising separating the fraction of step e) by Hydroxyapaptite column chromatography.

20. A pharmaceutically active fraction obtainable from the process of 19.

21. The fraction of claim 20, further comprising an agent selected from the group consisting of anti-angiogenic, anti-tumor or immune enhancing.

22. A method for inhibiting the growth of endothelial cells comprising delivering to the cells an effective amount of an active agent selected from the group consisting of ECTa, ECTb, ATC07α, ATC07β, ATC07β1, and ATC07β2.

23. The method of claim 22, further comprising delivering to the cells an effective amount of an agent selected from the group consisting of anti-angiogenic, anti-tumor or immune enhancing.

24. A method of inhibiting vascularization in a tissue comprising delivering to the tissue an effective amount of an active agent selected from the group consisting of ECTa, ECTb, ATC07α, ATC07β, ATC07β1, and ATC07β2.

25. The method of claim 24, further comprising delivering to the cells an effective amount of an agent selected from the group consisting of anti-angiogenic, anti-tumor or immune enhancing.

26. A method of treating a disorder associated with pathological neovascularization or endothelial cell growth in a subject, comprising administering to a subject a therapeutically effective amount of an active agent selected from the group consisting of ECTa, ECTb, ATC07α, ATC07β, ATC07β1, and ATC07β2.

27. The method of claim 26, further comprising delivering to the cells an effective amount of an agent selected from the group consisting of anti-angiogenic, anti-tumor or immune enhancing.

28. The method of claim 26, wherein the disorder is selected from the group Gus consisting of cancer, arthritic conditions, neovascular-based dermatological conditions, diabetic retinopathy, Karposi's Sarcoma, age-related macular degeneration, restenosis, telangectasia, glaucoma, keloids, corneal graft rejection, wound granularization, angiofibroma, Osler-Webber Syndrome, myocardial angiogenesis and scleroderma.

29. The method of claim 28, wherein the disorder is an arthritic condition selected from the group consisting of rheumatoid arthritis, psoriatic arthritis and osteoarthritis.

30. The method of claim 26, wherein the subject is an animal.

31. The method of claim 30, wherein the animal is selected from the group consisting of a pet, a farm animal or a human patient.

32. A method of treating a disorder associated with pathological neovascularization or endothelial cell growth in a subject, comprising administering to a subject a therapeutically effective amount of agent selected from the group consisting of ECTa, ECTb, ATC07α, ATC07β, ATC07β1, and ATC07β2.

33. The method of claim 30, further comprising delivering to the cells an effective amount of an agent selected from the group consisting of anti-angiogenic, anti-tumor or immune enhancing.

34. A method for screening for a therapeutic agent for inhibiting neovascularization or endothelial cell growth comprising the steps of:
a) contacting the agent with a suitable cell or tissue sample;
b) contacting a separate sample of the suitable cell or tissue sample with a therapeutically effective amount of an agent selected from the group consisting of ECTa, ECTb, ATC07α, ATC07β, ATC07β1, and ATC07β2; and
c) comparing the growth of the sample of step (a) with the growth of the sample of step (b), and wherein any agent of step (a) that inhibits the growth to the same or similar extent as the sample of step (b) is a therapeutic agent for inhibiting neovascularization or the growth of endothelial cells.

35. The method of claim 34, wherein the therapeutically effective amount of the extract of step (b) can further comprising delivering or administering an anti-tumor therapy or agent.

36. A kit comprising at least one agent selected from the group consisting of ECTa, ECTh, ATC07α, ATC07β, ATC07β1, and ATC07β2 and instructions for use.
